(19) United States
(12) Patent Application Publication
(10) Pub. No.: US 2020/0222404 A1
(43) Pub. Date: Jul. 16, 2020

(54) NANOHYBRID DRUG CARRIER PREPARED BY PICKERING EMULSION TEMPLATE METHOD WITH MAGADIITE AS EMULSIFIER AND PREPARATION METHOD THEREFOR

(71) Applicant: SOUTH CHINA UNIVERSITY OF TECHNOLOGY, Guangdong (CN)

(72) Inventors: Mingliang GE, Guangdong (CN); Luoxiang CAO, Guangdong (CN)

(73) Assignee: SOUTH CHINA UNIVERSITY OF TECHNOLOGY, Guangdong (CN)

(21) Appl. No.: 16/615,132

(22) PCT Filed: Dec. 1, 2017
(86) PCT No.: PCT/CN2017/114187

§ 371 Date: Nov. 20, 2019

(30) Foreign Application Priority Data
Mar. 29, 2017 (CN) 201710199353.2

(51) Int. Cl. A61K 31/513; A61K 31/57; A61K 31/704; A61K 9/00; A61K 9/51

(52) U.S. Cl. CPC A61K 31/513; A61K 31/57; A61K 9/5153; A61K 9/0002; A61K 9/5123; A61K 31/704

(57) ABSTRACT

A nanohybrid drug carrier prepared by a Pickering emulsion template method with magadiite as an emulsifier and a preparation method therefor. With organic magadiite as the emulsifier and an organic solvent capable of dissolving and dispersing a PLGA as an oil phase, a Pickering drug emulsion is prepared, and then a PLGA-magadiite nanohybrid drug controlled-release carrier is prepared by using a solvent evaporation method.

### BACKGROUND OF THE INVENTION

### 1. Technical Field

The present invention relates to the field of drug carriers, and more particularly, to a nanohybrid drug carrier prepared by a Pickering emulsion template method with magadiite as an emulsifier and a preparation method.

### 2. Background Art

Traditional drugs have no controlled-release property and tissue specificity, and large amount of drugs are released at the initial stage after administration, which brings harm to a body; and systemic drug release also causes damages to normal organs and tissues. Meanwhile, the body decomposes and excretes the drugs quickly, which results in that a drug concentration can reach a certain therapeutic level only after repeated administration. This is especially common in the chemotherapy process of tumor patients. Therefore, it is of great practical significance to treat patients to make these drugs into sustained-release preparations, protect the structures of the drugs from being damaged by surrounding environments, maintain the due efficacy of the drugs, reduce the number of times of taking drugs, improve the bioavailability, reduce the toxic and side effects of the drugs, or the like. Biodegradable nano drug carriers have become research hotspots due to their advantages of slowing down drug degradation, reducing drug phagocytosis by a reticuloendothelial phagocytosis system, improving the bioavailability, prolonging in vivo circulation time and improving cell permeability, etc. A polylactic acid-glycolic acid copolymer PLGA, which has excellent biocompatibility, can be degraded in vivo, is non-toxic and harmless to the body, can delay the drug release time, improve the half-life period of the drugs and reduce the drug toxicity, is widely applied to nano drug controlled-release carriers. However, the industrial application of the PLGA is limited by drug burst release. It has been reported that the burst release is controlled by controlling particle sizes of nanomicrospheres, modifying structure of microspheres, combining polymers, selecting additives and other methods.

An emulsion is a thermodynamically unstable system. An emulsifier must be added to the system to obtain a stable emulsion by reducing an interfacial tension between two phases. However, excessive non-food grade traditional emulsifier must be removed from a sample, otherwise the emulsifier will cause harm to a human body and affect and destroy the subsequent application of the emulsion. For example, the emulsifier can induce tissue inflammation and even cause cell damage, which limits the application of the traditional emulsion prepared from the emulsifier in medical preparations. Solid particles can prevent emulsion droplets from coalescence through an interfacial effect, thus replacing the traditional emulsifier to prepare a stable Pickering emulsion. Solid magadiite can be synthesized by using pure chemical reagents as raw materials, thus obtaining high-purity and stable products, and the raw materials for synthesizing the magadiite have wide sources, low price and low cost. The magadiite is nontoxic and does not produce adverse reactions to the human body. Clay and drugs may interact with each other, and clay with similar structures has been applied to a drug controlled-release system. In the present invention, the magadiite is used as an emulsifier to prepare a stable Pickering emulsion, and a PLGA-magadiite nanohybrid drug carrier is prepared by using an evaporation method. By making full use of the advantages of the Pickering emulsion like no pollution, environmental friendliness, little toxic effect on the human body, strong stability and the like, and adjusting the concentration of the nanoparticle emulsifier or the oil-water ratio of the emulsion, the size of the emulsion particles is adjusted and controlled, and the drug loading efficiency is improved.

### SUMMARY OF THE INVENTION

The present invention aims to provide a nanohybrid drug carrier prepared by a Pickering emulsion template method with magadiite as an emulsifier and a preparation method therefor. The drug carrier comprises an organic magadiite, a poly(lactic-co-glycolic acid) (PLGA) and a drug.

The object of the present invention is achieved by the following technical solutions.

A method for preparing a nanohybrid drug carrier by a Pickering emulsion template method with magadiite as an emulsifier comprises the following steps:

1) adding a PLGA, a model drug and an organic magadiite with a contact angle θ of less than 90° into an oil phase, mechanically stirring and ultrasonically dispersing the same to be even to obtain a mixture A;

2) adding the mixture A into deionized water, stirring and ultrasonically dispersing the mixture A to obtain a stable O/W Pickering emulsion; and

3) heating up to evaporate an organic solvent of an internal phase by using a solvent evaporation method firstly, so that the PLGA is slowly precipitated and solidified to form a nanohybrid with magadiite and the drug is contained in the hybrid, and then drying to remove water to obtain a nanohybrid drug carrier which is a PLGA-magadiite nanohybrid drug controlled-release microsphere.

Preferably, the model drug is a water-insoluble drug, and a volume ratio of oil to water in step 2) is less than 1.

Preferably, the model drug is levonorgestrel or paclitaxel.

Preferably, the oil phase is methylene chloride or ethyl acetate.

Preferably, the organic magadiite is obtained by modifying magadiite with one of organic quaternary phosphonium salt (phosphonium refers to PH4⁺), organic quaternary ammonium salt and silane.

Preferably, the PLGA, the model drug, the organic magadiite.

A method for preparing a nanohybrid drug carrier by a Pickering emulsion template method with magadiite as an emulsifier comprises the following steps:

1) adding a PLGA and an organic magadiite with a contact angle θ of greater than 90° into an oil phase, mechanically stirring and ultrasonically dispersing the same to be even to obtain a mixture B;

2) adding the mixture B into deionized water dissolved with a model drug, stirring violently and ultrasonically dispersing the mixture B to obtain a stable W/O Pickering emulsion; and

3) evaporating an organic solvent of an external phase by using a solvent evaporation method, so that the PLGA is precipitated to form a nanohybrid with magadiite and the drug is contained in the hybrid, and then heating up and drying to remove water in the internal phase to obtain a nanohybrid drug controlled-release carrier which is a PLGA-magadiite nanohybrid drug controlled-release membrane.

Preferably, the model drug is a water-soluble drug, and a volume ratio of oil to water in step 2) is greater than 1.

Preferably, the model drug is doxorubicin.

Preferably, the oil phase is methylene chloride or ethyl acetate.

Preferably, the organic magadiite is obtained by modifying magadiite with one of organic quaternary phosphonium salt, organic quaternary ammonium salt and silane.

Preferably, the PLGA, the model drug, the organic magadiite.

A nanohybrid drug carrier prepared by the method above.

Preferably, the nanohybrid drug carrier comprises the following components: an organic magadiite, a PLGA and a drug.

The nanohybrid drug carrier comprises two structures, which are a PLGA-magadiite nanohybrid drug controlled-release microsphere prepared by an O/W Pickering emulsion template method and a PLGA-magadiite nanohybrid drug controlled-release membrane prepared by a W/O Pickering emulsion template method.

According to the invention, the organic magadiite is used as the emulsifier, and the organic solvent capable of dissolving and dispersing the PLGA is used as the oil phase to prepare the Pickering drug emulsion, and then the PLGA-magadiite nanohybrid drug controlled-release carrier is prepared by using the solvent evaporation method. By adding organic magadiite with different hydrophile-lipophile balance values, an oil/water interface is adjusted to have different three-phase contact angles θ, and two nanohybrid drug carriers with different structures are prepared: the PLGA-magadiite nanohybrid drug controlled-release microsphere prepared by the O/W Pickering emulsion template method and the PLGA-magadiite nanohybrid drug controlled-release membrane prepared by the W/O Pickering emulsion template method.

The drug carrier of the present invention has the advantages of slowing down drug degradation, reducing drug phagocytosis by a reticuloendothelial phagocytosis system, improving the bioavailability, prolonging in vivo circulation time, and improving cell permeability, etc. The Pickering emulsion is used as the template, and compared with the traditional emulsion, the Pickering emulsion has the advantages of no pollution, environmental friendliness, less toxic effect on the human body, strong stability, and the like. By adjusting the concentration of the nanoparticle emulsifier or the oil-water ratio of the emulsion, the size of the emulsion particles is adjusted and controlled, and the drug loading efficiency is improved. The present invention has important research values and application values in such fields as biology, medicine, materials and the like, which are related to drug carriers, controlled-release materials, catalyst carriers, etc.

Compared with the prior art, the present invention has the following advantages:

1. the nanohybrid drug carrier prepared by the present invention is a microsphere or a porous membrane structure, and the preparation process has mild conditions, and is simple and convenient to operate;

2. the present invention adopts the organic magadiite as the emulsifier, and can obtain organic magadiite with different three-phase contact angle θ values by modifying magadiite with different organic reagents, so as to prepare the O/W and W/O stable Pickering emulsions; and

3. the present invention adopts the PLGA polymer which can be biodegraded in vivo and in vitro, has no toxic effect on the body, and has good compatibility with the human body.

### BRIEF DESCRIPTION OF THE DRAWINGS

FIG. 1 is a microscopic schematic diagram of a process of preparing a PLGA-magadiite nanohybrid drug controlled-release microsphere;

FIG. 2 is a microscopic schematic diagram of a process of preparing a PLGA-magadiite nanohybrid drug controlled-release membrane;

FIG. 3a is a SEM graph of pure magadiite;

FIG. 3b is a SEM graph of magadiite modified by cetyltriphenyl quaternary phosphonium salt;

FIG. 4 is a polarization microscope graph of an emulsion; and

FIG. 5 is a TEM graph of a PLGA-magadiite encapsulating drug 5-fluorouracil.

### DETAILED DESCRIPTION OF THE EMBODIMENTS

The concrete implementation of the invention is further described hereinafter with reference to the accompanying drawings and embodiments, but the invention is not limited thereto.

### Embodiment 1

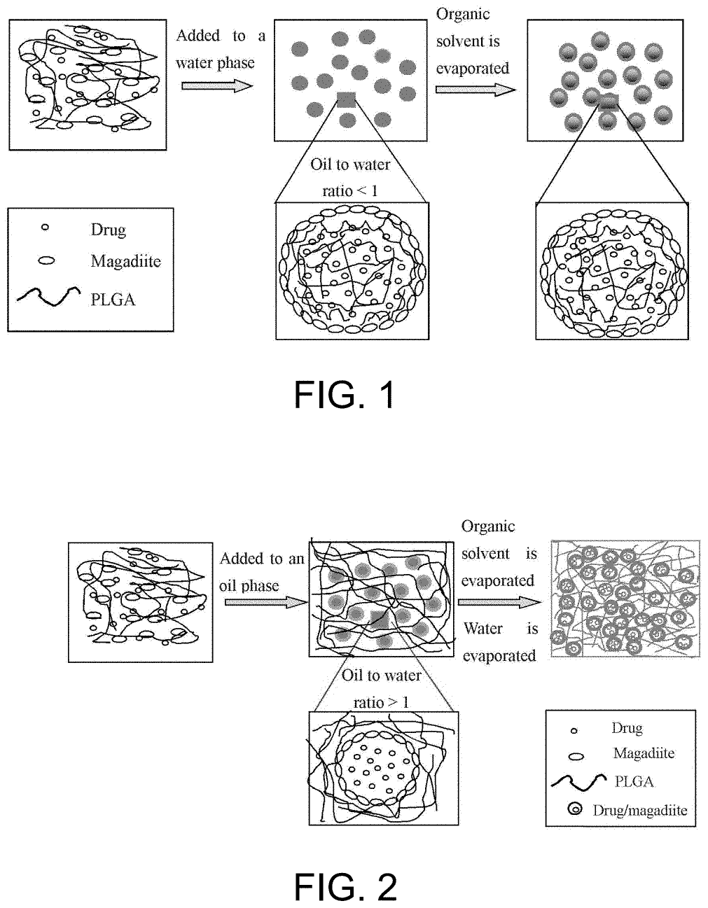

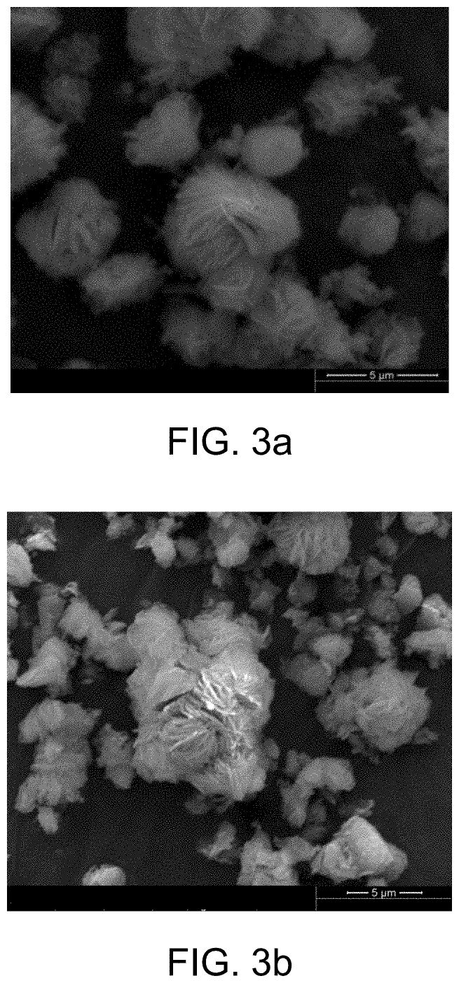

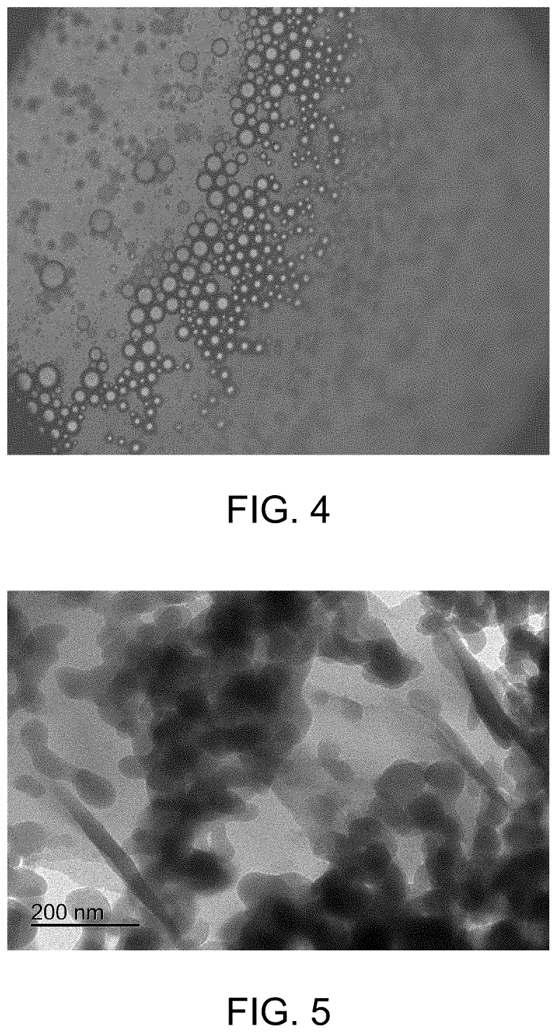

5 g of magadiite (see FIG. 3a for SEM graph) and 1 g of cetyltriphenyl phosphonium bromide were weighed and put into a 500 ml beaker, and then added with 100 ml of deionized water to form a mixture. The beaker was placed in a magnetic stirring water bath kettle and the mixture was stirred for 24 hours at 80° C. After the reaction was completed, a product was filtered and washed for three times with deionized water. A resulting filtrate was dried for 6 hours at 80° C., and then grinded to obtain organic magadiite (see FIG. 3b for SEM graph). 2 mg of levonorgestrel, 1 g of organic magadiite and 1 g of PLGA were weighed, mixed and dissolved in 50 ml of ethyl acetate to form a mixture which was then placed in a 100 ml beaker after mixing. After the beaker was placed in a magnetic stirring water bath kettle and the mixture was stirred for 6 hours at room temperature, the beaker was placed in an ultrasound environment (40 KHz) for 3 hours, and then the mixture was added into deionized water, wherein a volume ratio of water to oil was 1:2. A stable and even milky Pickering emulsion (see FIG. 4 for polarization microscope graph) was obtained by ultrasonic mixing, and then the oil phase ethyl acetate was removed by using a solvent evaporation method, and finally the emulsion was dried at 80° C. in vacuum to obtain a nanohybrid drug controlled-release microsphere encapsulating drug levonorgestrel. The structural diagram of the nanohybrid drug controlled-release microsphere is shown in FIG. 1. The modified organic magadiite acts as a hard phase to support and is wound with the PLGA to form the microsphere in which the drug was contained.

### Embodiment 2

5 g of magadiite and 1 g of cetyltrimethyl ammonium bromide were weighed and put into a 500 ml beaker, and then added with 100 ml of deionized water to form a mixture. The beaker was placed in a magnetic stirring water bath kettle and the mixture was stirred for 24 hours at 80° C. After the reaction was completed, a product was filtered and washed for three times with deionized water. A resulting filtrate was dried for 6 hours at 80° C., and then grinded to obtain organic magadiite. 1 g of organic magadiite and 1 g of PLGA were weighed, mixedly and dissolved in 50 ml of methylene chloride to form a mixture which was placed in a 100 ml beaker after ultrasonic mixing. 2 mg of doxorubicin was dissolved in 50 ml of deionized water, an oil phase was mixed with an aqueous phase, a volume ratio of water to oil was 2:1, and then the mixture was placed in an ultrasound environment (40 KHz) for 3 hours to obtain a stable and milky Pickering emulsion. After that, the oil phase methylene chloride was removed by using a solvent evaporation method, and finally the emulsion was dried at 80° C. in vacuum to obtain a nanohybrid drug controlled-release membrane containing drug doxorubicin. The structural diagram of the nanohybrid drug controlled-release membrane is shown in FIG. 2.

### Embodiment 3

5 g of magadiite and 1 g of cetyltrimethyl quaternary phosphonium salt were weighed and put into a 500 ml beaker, and then added with 100 ml of deionized water to form a mixture. The beaker was placed in a magnetic stirring water bath kettle and the mixture was stirred for 24 hours at 80° C. After the reaction was completed, a product was filtered and washed for three times with deionized water. A resulting filtrate was dried for 6 hours at 80° C., and then grinded to obtain organic magadiite. 2 mg of 5-fluorouracil, 1 g of organic magadiite and 1 g of PLGA were weighed, mixed and dissolved in 50 ml of ethyl acetate to form a mixture which was then placed in a 100 ml beaker after even ultrasonic mixing. After the beaker was placed in a magnetic stirring water bath kettle and the mixture was stirred for 6 hours at room temperature, the beaker was placed in an ultrasound environment (40 KHz) for 3 hours, and then the mixture was added into deionized water, wherein a volume ratio of water to oil was 8:9. A stable and even milky Pickering emulsion was obtained by ultrasonic mixing, then the oil phase ethyl acetate was removed by using a solvent evaporation method, and finally the emulsion was dried at 80° C. in vacuum to obtain a nanohybrid drug controlled-release microsphere encapsulating drug 5-fluorouracil. FIG. 5 is a TEM graph of PLGA-magadiite encapsulating drug 5-fluorouracil.

The above-mentioned embodiments of the invention are merely examples for clearly illustrating the invention and are not intended to limit the embodiments of the invention. For those of ordinary skills in the art, other different forms of changes or variations can be made on the basis of the above description. It is not necessary or possible to exhaust all the embodiments here. Any change, equivalent substitution, and improvement made within the spirit and principle of the invention shall fall within the protection scope of the claims of the invention.

1. A method for preparing a nanohybrid drug carrier by a Pickering emulsion template method with magadiite as an emulsifier, wherein comprising the following steps:
1) adding a PLGA, a model drug and an organic magadiite with a contact angle θ of less than 90° into an oil phase, mechanically stirring and ultrasonically dispersing to even to obtain a mixture A;
2) adding the mixture A into deionized water, stirring and ultrasonically dispersing to obtain a stable O/W Pickering emulsion; and
3) by using a solvent evaporation method, heating up to evaporate an organic solvent of an internal phase, followed by drying to remove water to obtain a nanohybrid drug carrier, which is a PLGA-magadiite nanohybrid drug controlled-release microsphere.

2. A method for preparing a nanohybrid drug carrier by a Pickering emulsion template method with magadiite as an emulsifier, wherein comprising the following steps:
1) adding a PLGA and an organic magadiite with a contact angle θ of greater than 90° into an oil phase, mechanically stirring and ultrasonically dispersing to even to obtain a mixture B;
2) adding the mixture B into deionized water dissolved with a model drug, stirring and ultrasonically dispersing to obtain a stable W/O Pickering emulsion; and
3) by using a solvent evaporation method, evaporating an organic solvent of an external phase, followed by heating up and drying to remove water in an internal phase to obtain a nanohybrid drug controlled-release carrier which is a PLGA-magadiite nanohybrid drug controlled-release membrane.

3. The method according to claim 1, wherein the model drug is a water-insoluble drug, and a volume ratio of oil to water in the step 2) is less than 1.

4. The method according to claim 2, wherein the model drug is a water-soluble drug, and a volume ratio of oil to water in the step 2) is greater than 1.

5. The method according to claim 1, wherein the model drug is levonorgestrel or paclitaxel.

6. The method according to claim 2, wherein the model drug is doxorubicin.

7. The method according to claim 1, wherein the oil phase is methylene chloride or ethyl acetate.

8. The method according to claim 1, characterized in that, wherein the organic magadiite is obtained by modifying a magadiite with one of an organic quaternary phosphonium salt, an organic quaternary ammonium salt and a silane.

9. A nanohybrid drug carrier prepared by the method according to claim 1.

10. The nanohybrid drug carrier according to claim 9, wherein the nanohybrid drug carrier comprises the following components: an organic magadiite, a PLGA and a drug.

11. The method according to claim 2, wherein the oil phase is methylene chloride or ethyl acetate.

12. The method according to claim 2, wherein the organic magadiite is obtained by modifying a magadiite with one of an organic quaternary phosphonium salt, an organic quaternary ammonium salt and a silane.

13. A nanohybrid drug carrier prepared by the method according to claim 2.

14. The nanohybrid drug carrier according to claim 13, wherein the nanohybrid drug carrier comprises the following components: an organic magadiite, a PLGA and a drug.

15. A nanohybrid drug carrier prepared by the method according to claim 3.

16. The nanohybrid drug carrier according to claim 15, wherein the nanohybrid drug carrier comprises the following components: an organic magadiite, a PLGA and a drug.

17. A nanohybrid drug carrier prepared by the method according to claim 4.

18. The nanohybrid drug carrier according to claim 17, wherein the nanohybrid drug carrier comprises the following components: an organic magadiite, a PLGA and a drug.

19. A nanohybrid drug carrier prepared by the method according to claim 8.

20. The nanohybrid drug carrier according to claim 19, wherein the nanohybrid drug carrier comprises the following components: an organic magadiite, a PLGA and a drug.
